(19) United States
(12) Patent Application Publication
(10) Pub. No.: US 2025/0152096 A1
(43) Pub. Date: May 15, 2025

(54) CALIBRATED WEARABLE VITAL SIGNS MONITORING SYSTEM AND METHOD

(71) Applicant: NotYet Inc., Wilmington, DE (US)

(72) Inventors: Elena Dobrokhotova, Netanya (IL); Vladimir Zeldets, Beer Sheva (IL); Polina Molodtsova, Netanya (IL); Ramil Teregulov, Edirne Merkez/Edirne (TR)

(21) Appl. No.: 18/508,774

(22) Filed: Nov. 14, 2023

(51) Int. Cl. A61B 5/00; A61B 5/0205; A61B 5/022; A61B 5/1455; A61B 5/256; A61B 5/282

(52) U.S. Cl. CPC A61B 5/486; A61B 5/02055; A61B 5/02225; A61B 5/14552; A61B 5/256; A61B 5/282; A61B 5/681; A61B 5/7246; A61B 5/7275; A61B 5/741; A61B 5/744; A61B 2562/0219

(57) ABSTRACT

A calibrated system for measuring and monitoring vital signs of a user. The system provides for a wearable device. The wearable device comprises an optical module, an oscillometric module and an electrocardiogram (ECG) module, at least one processor, and a non-transitory machine-readable medium comprising instructions stored therein. The processor performs the following operations: establishing a user initial profile based on an initial information, determining a user baseline profile from the initial information, vital signs measurements received from the wearable device for a time period, determining a user reference model based on the user baseline profile, the user reference model comprises zone indicators, receiving actual vital signs measurements in real time from the wearable device, comparing the actual vital signs measurements with the user reference model, interpreting whether a result of the comparison between the actual vital signs measurements and the user reference model is within predetermined parameters, and providing a feedback based on the interpretation of the result of the comparison between the actual vital signs measurements and the user reference mode.

### BACKGROUND

The present invention generally relates to wearable devices for monitoring vital signs. More specifically, the present invention relates to a real-time calibrated system with a wearable device for monitoring vital signs, such as skin temperature, blood pressure, heart rate, pulse rate, respiration rate, and functional oxygen saturation.

Generally, vital signs are objective measures of physiological function that are used to monitor acute and chronic disease, including cardiovascular diseases, and thus serve as a basic communication tool about a person's health status. The traditional vital signs include skin temperature, blood pressure, heart rate, pulse rate, and functional oxygen saturation. The vital signs can identify and signal molecular changes, organ system changes, systemic changes, and altered compensation to stressors in a person.

More specifically, in older persons, as a result of age or age-associated pathophysiology, coupled with age-related loss of protective homeostatic mechanisms, vital sign response may deviate from standard ranges for a specific patient, and remain confined to a range of values, unable to respond appropriately to environmental stressors.

Because of the tendency to reduce homeostatic mechanisms with age, difficulty maintaining internal consistency prevents the optimal function of the body and hinders its ability to respond to specific homeostatic challenges. Thus, a service provider (e.g., clinician) is less dependent on normative ranges for the overall population, but more dependent on normal ranges for the specific older person. As a result, successive vital sign measurements for an individual are more sensitive to change because an inconsistent vital sign indicates that a negative health event is significant enough to exceed the threshold of the confined range for the person.

Moreover, accurate vital signs, especially blood pressure, are more difficult to obtain in older population, for example, due to loss of elasticity in vein walls, especially when using optical blood pressure monitoring.

A solution is therefore needed that allows for systematic vital signs measurements that are dependent on vital signs ranges of the specific older person, taking into account such persons clinical data, rather than overall population. It is also desirable that the vital signs measurements are verifiable via continuous calibration without the use of external devices. Moreover, it is desirable for the solution to be easily accessible outside of a professional setting (at home) for older population.

### SUMMARY

According to a non-limiting embodiment of the present invention, a calibrated system for measuring and monitoring vital signs of a user. The system provides for A calibrated system for measuring and monitoring vital signs of a user. The system provides for a wearable device. The wearable device has an optical module, an oscillometric module and an electrocardiogram (ECG) module, at least one processor, and a non-transitory machine-readable medium comprising instructions stored therein. The processor is configured to establish a user initial profile based on an initial information, determine a user baseline profile from the initial information, vital signs measurements received from the wearable device for a time period, determine a user reference model based on the user baseline profile, the user reference model comprises zone indicators, receives actual vital signs measurements in real time from the wearable device, compare the actual vital signs measurements with the user reference model, interpret whether a result of the comparison between the actual vital signs measurements and the user reference model is within predetermined parameters, and provide a feedback based on the interpretation of the result of the comparison between the actual vital signs measurements and the user reference mode.

In another aspect of the present invention a method for monitoring vital signs using a wearable device is provided. The wearable device has an optical module, an oscillometric module and an electrocardiogram (ECG) module, at least one processor, and a non-transitory machine-readable medium comprising instructions stored therein, which when executed by the processor can have the following steps: establishing a user initial profile based on an initial information, determining a user baseline profile from the initial information, vital signs measurements received from the wearable device for a time period, determining a user reference model based on the user baseline profile, the user reference model comprises zone indicators, receiving actual vital signs measurements in real time from the wearable device, comparing the actual vital signs measurements with the user reference model, interpreting whether a result of the comparison between the actual vital signs measurements and the user reference model is within predetermined parameters, and providing a feedback based on the interpretation of the result of the comparison between the actual vital signs measurements and the user reference mode.

In yet another aspect of the present invention the wearable device can be calibrated by the following steps: determining a rest activity state of the user, establishing an assigned position of the wearable device, assigning a validation period, determining a first blood pressure measurement by the oscillometric module, determining a second blood pressure measurement by the optical module, comparing the first blood pressure measurement to the second blood pressure measurement, and if the second blood pressure measurement is measurably different from the first blood pressure measurement, calibrating the optical module based on the first blood pressure measurement.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts an exemplary diagram of a calibrated system for vital signs measurements in accordance with embodiments of this invention;

FIG. 2 depicts a diagram of an exemplary wearable device in accordance with embodiments of this invention;

FIG. 3 depicts a diagram of a computer program in accordance with embodiments of this invention;

FIG. 4 depicts a diagram of a calibrations steps for blood pressure monitoring of the wearable device in accordance with embodiments of this invention; and

FIG. 5 depicts a diagram of an exemplary embodiment of the event flow of a method for vital signs measurements in accordance with embodiments of this invention in accordance with embodiments of this invention of this invention.

In the accompanying figures and following detailed description of the described embodiments, the various elements illustrated in the figures are provided with two, three or four digit reference numbers. With minor exceptions, the leftmost digit(s) of each reference number correspond to the figure in which its element is first illustrated.

### DETAILED DESCRIPTION

Reference to “a specific embodiment” or a similar expression in the specification means that specific features, structures, or characteristics described in the specific embodiments are included in at least one specific embodiment of the present invention. Hence, the wording “in a specific embodiment” or a similar expression in this specification does not necessarily refer to the same specific embodiment.

Hereinafter, various embodiments of the present invention will be described in more detail with reference to the accompanying drawings. Nevertheless, it should be understood that the present invention could be modified by those skilled in the art in accordance with the following description to achieve the excellent results of the present invention. Therefore, the following description shall be considered as a pervasive and explanatory description related to the present invention for those skilled in the art, not intended to limit the claims of the present invention.

Reference to “an embodiment,” “a certain embodiment” or a similar expression in the specification means that related features, structures, or characteristics described in the embodiment are included in at least one embodiment of the present invention. Hence, the wording “in an embodiment,” “in a certain embodiment” or a similar expression in this specification does not necessarily refer to the same specific embodiment.

Embodiments of the present invention are directed to a real-time calibrated wearable device for monitoring vital signs. The traditional vital signs include blood pressure, pulse rate, heart rate, respiratory rate, and skin temperature. The vital signs are objective measures of physiological function and are used to monitor acute and chronic disease, including cardiovascular diseases. Thus, the vital signs can identify and signal a negative health event or organ system changes in a person.

It is therefore important for at risk persons, such as the older population, to have continues access to the vital signs to signal a negative health event or organ system change. However, in older persons, as a result of age or age-associated pathophysiology, for example, a loss of elasticity in vessels walls, vital sign responses may deviate from normal ranges, and at times unable to respond appropriately to environmental stressors. Moreover, in older population, to determine deviations for each vital sign indicator, many factors need to be take into account, such as activity level, diseases, anamnesis, and ongoing medical treatment. Standards for older population are most often too “broad” and do not reflect individual medical characteristics acquired over a lifetime.

Generally, the available vital sign monitors offered outside of healthcare facilities settings (e.g., hospitals) provide vital sign monitoring does not account for deviation in the vital sign responses in the older population. These devices are often limited to the data the devices receive from the user and do not account for activity level, diseases, anamnesis, and ongoing medical treatment of the user. More importantly, the available vital sign monitoring do not offer calibration of real-time measurements, and thus often provide less than precise measurements and/or require calibration in an outside facility.

In order to address the forgoing shortcomings, embodiments of the present invention provide a system that allows for systematic and ongoing vital signs measurements that are dependent on normal ranges of the specific older person, rather than overall population, and that are verifiable via continuous calibration.

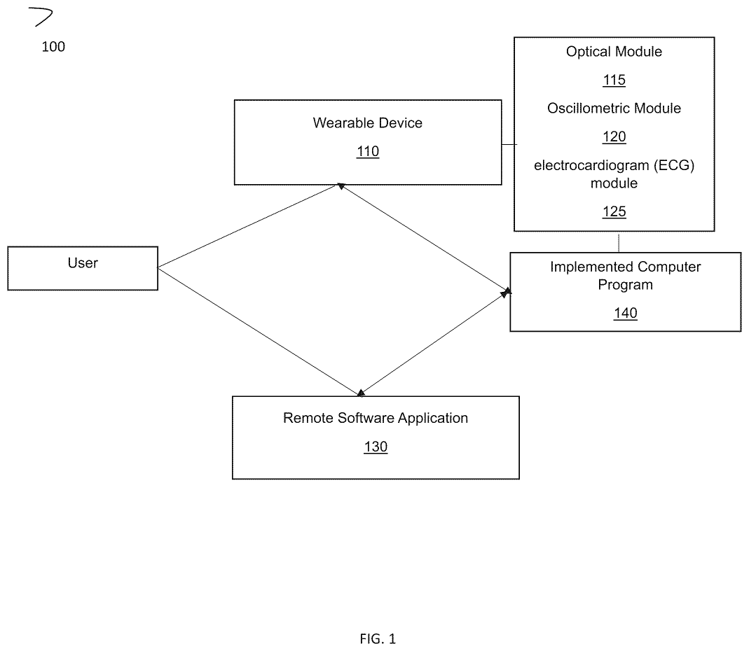

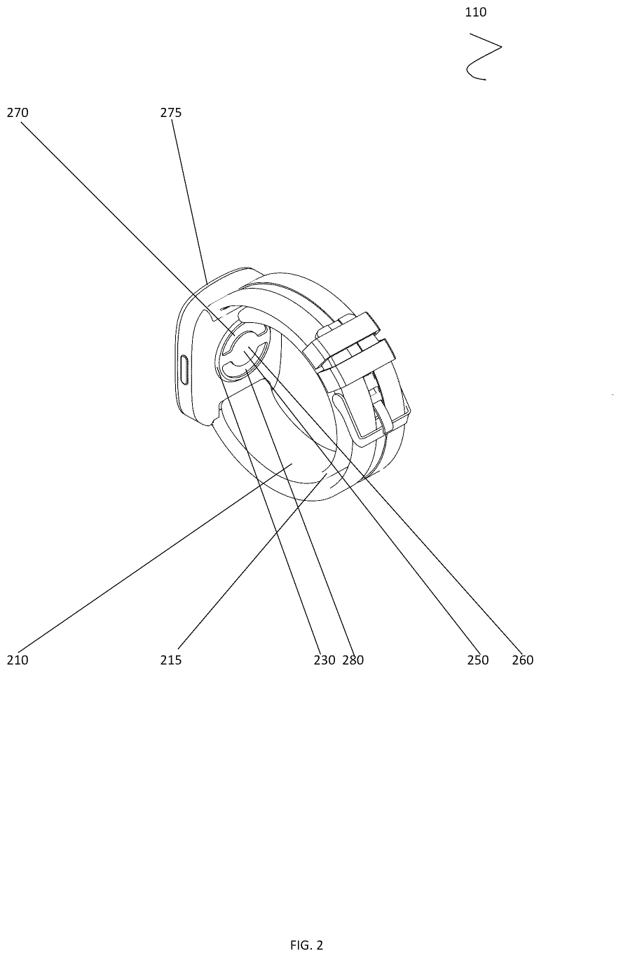

FIG. 1 illustrates an exemplary embodiment of a system 100 that has a wearable device 110 (shown in detail on FIG. 2). The wearable device 110 can include an optical module 115, an oscillometric module 120 and an electrocardiogram (ECG) module 125. The system 100 can further have a remote software application 130 for use on a portable device, such as the user's mobile phone or tablet, or for use on a non-portable device, such as a desktop computer. The wearable device 110 and the remote software application 130 are connected to an implemented computer program 140 that operates one or more modules remotely via cloud computing services accessible via network connection. That is, the device 110 can be connected over a network to one or more servers (not shown).

FIG. 2 illustrate the wearable device 110 in a close position in accordance with the embodiments of the preset invention. The oscillometric module 120 of the wearable device is configured to use oscillometric method to measure blood pressure of the user. The oscillometric module 120 can include a cuff 210 configured to inflate using a pump 215 and deflate using an electric valve (not shown). The pump 215 can be piezoelectric pump. The oscillometric module 120 also can have a pump driver to operate the pump 215. The oscillometric module 120 has a pressure sensor 230 to determine a systolic and diastolic blood pressure of the user. Using the oscillometric module 120, the user can measure his or her blood pressure at any desired time. Any oscillometric blood pressure measurement principle, which is employed by most cuff devices, and associated algorithms can be used. For example, the external pressure (Pe) of an artery is swept via cuff inflation/deflation, and Pe (i.e., cuff pressure) is measured via pressure sensor 230 and high-pass filtered to yield oscillations (O). Systolic BP (Ps), mean BP (Pm), and/or diastolic BP (Pd) are then estimated from the oscillogram [peak-to-peak amplitude or envelope difference of the oscillation (0) vs. Pe function] via an empirical algorithm. Popular algorithms include the maximum amplitude, derivative, and fixed ratio algorithms. The blood pressure measurement obtained by this method using the oscillometric module 120 is then sent and stored in non-volatile memory (NVM) of the implemented computer program 140 as a first blood pressure measurement.

The optical module 115 is configured to measure the blood pressure of the user without the use of the oscillometric module 120 (cuffless), but rather using the principles of pulse transmission time (PTT), generally, defined as the transit time for a pressure pulse from heartbeat of the user's arterial system, which correlates with both systolic and diastolic blood pressure. The optical module can have an optical sensor array 250 and light-emitting diode (LED) 260 operating near 570 nm, a photodetector (not shown). The optical sensor array 250 is provided on the bottom side of the wearable device 110 (as shown in FIG. 2). The LED 260 transmits a light into the user's skin and part of this light is reflected from the tissue and detected by the photodetector. According to embodiments of the present invention, a second blood pressure measurement is measured by using optical module 115 via linear regression analysis.

The optical module 115 also allows the measurements of arterial oxygenation (SPO2%), respiration rate, pulse rate, heart rate, and to calculate heart rate variability (HRV).

According to embodiment of the present invention, the ECG module 125 can include two contact electrodes 270, 275. The first electrode 270 is the sensor electrode located on the bottom side of the wearable device 110 and is in contact with the wrist of the user. The second electrode 275 (which is also a sensor) is located on the front side of the wearable device 110.

According to embodiment of the present invention, the measurement can be performed by the user contacting the second electrode 275 with the palm, and the opposite hand to complete the circuit across the heart. The ECG measurement can provide a rhythm classification and an ECG graph of a single channel electrocardiogram (ECG) similar to a Lead I ECG. For example, the ECG module 125 can measure Low Heart Rate (bradycardia) (<50 beats per minute (bpm)), Sinus Rhythm (50-99 bpm), High Heart Rate (tachycardia) (100-150 bpm), Atrial Fibrillation (50-99 bpm), Atrial Fibrillation High Heart Rate (100-150 bpm), and High Heart Rate (>150 bpm).

In addition, the wearable device 110 can have a temperature sensor 280 to measure skin temperature. The temperature sensor 280 preferably configured to be positioned on the bottom of the wearable device 110 and be in direct contact with the skin, and continuously measuring the skin.

The wearable device 110 can also have an advance positioning sensor 290 (accelerometer and/or gyroscope) to aid the user to determine if the wearable device 110 is at the correct height in relation to the heart during blood pressure measurement. The correct positioning greatly improves the accuracy of the measurement. This determination is based on the reading of an accelerometer (integral to the wearable device110) to measure the angle of the arm.

The advance positioning sensor 290 can also allow to determine the activity state of the user (walking, running and the like). The advance positioning sensor 290 can detect a fall of the user and signal for help. For example, in case of a fall detection the wearable device 110 sends a signal to the remote software application 130 that will transfer the signal to the server launching the process of a call from a call center and/or sending the information to the supervisor or the call center, depending if the user is subscribed to one of the applicable assistance programs.

Moreover, advance positioning sensor 290 can detect and/or allow the user to select which hand (left/right), the user uses for measurements, using the optical module 115, the oscillometric module 120 or the ECG module 125. The selection can be carried out by using remote software application 130. If the user changed the hand on which he or she is wearing the wearable device 110, and forgot to change the settings via the remote software application 130, the wearable device 110 is configured to automatically switch the algorithm (ECG) considering the hand selection, and will continue measurements without interruption. The ECG signal can be used to determine on which hand the user is wearing the wearable device 110.

Embodiments of the present invention also allow simultaneous measurement of blood pressure and ECG. To achieve this, the user must place his or her hand with the wearable device 110 on the chest while the opposite hand is placed on top of the wearable device. The wearable device 110 should be at heart level with the palm of the user's other hand pressed against the top of the wearable device 110 as determined by the advance positioning sensor 290. This allows measuring blood pressure, ECG, and PTT.

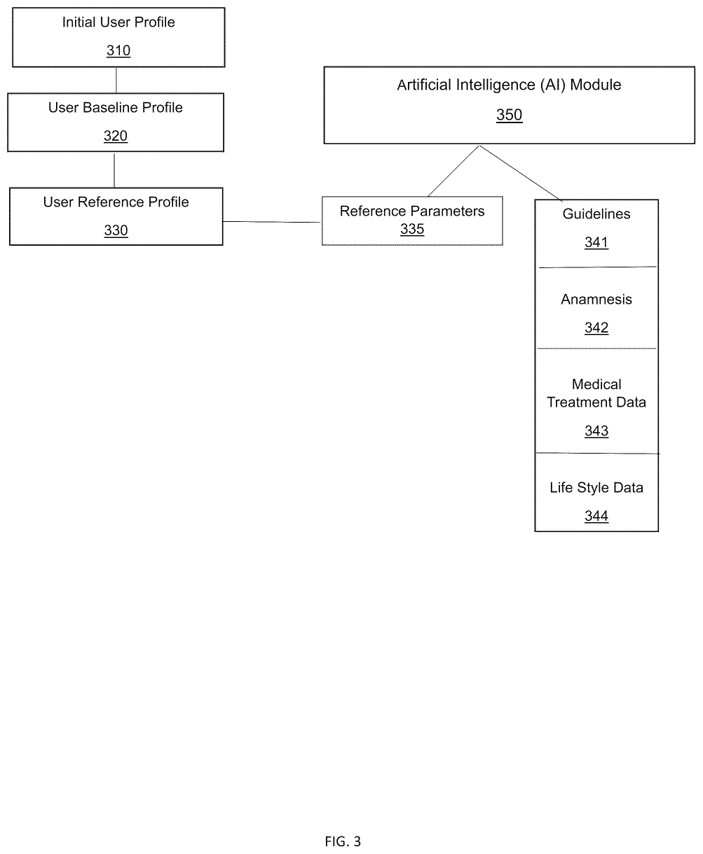

According to embodiment of the present invention, as illustrated in FIG. 3, the implemented computer program 140 detects a user initial profile 310 if the user previously established the user initial profile 310. Alternatively, the computer program 140 will prompt the user to establish the user initial profile 310. The user initial profile 310 can include the user's information, such as name, gender, age other non-medical information.

Based on the user initial profile 310, a user baseline profile 320 is established by including the initial vital signs measurements received from the wearable device 110. Preferably, the vital signs measurements have been taken for a set period of time, for example, seven days. The vital signs can be those measured by the optical module 115, the oscillometric module 120 or the ECG module 125 taken over a consecutive seven-day period, and include skin temperature, blood pressure, heart rate, pulse rate, respiration rate, and functional oxygen saturation. Preferably, the user baseline profile 310 is automatically updated every 15-30 days.

As further illustrated in FIG. 3, once the user baseline profile 320 has been established, the implemented computer program 140 is configured to determine a user reference model 330. The user reference model 330 is determined from the user baseline profile 320 by applying reference parameters 335. The reference parameters 335 can include guidelines 341 for vital parameters. In particular, the guidelines 341 include reference parameters to determine the acceptable limits of normal vital parameters in case there is no history of diseases that could influence the limits for each particular parameter. The reference parameters can be obtained from national health organizations, such as National Institute of Health (NIH), and the like. The guidelines 341 can also include abnormal vital parameters to adjust the limits of normal parameters depending on the presence of particular diseases.

The reference parameters can also have anamnesis 342 to account for which specific diseases affect the specific measured indicators. The anamnesis 342 allows to establish the user reference model 330 as accurately as possible for each particular user.

The reference parameters 335 can have medical treatment data 343. Data relating to medical treatment allows to estimate and control the normal range for certain indicators of user reference model 330 which can be influenced and controlled by a medication, when such indicators should have been out of a normal limit due to presence of a certain disease.

The reference parameters 335 can also have a life style data 344. The lifestyle data 344 can include body mass index (BMI), smoking and alcohol intake information, exercise regularity, and particulars of user's diet. The life style data 344 influence the limits of normal range for certain vital parameters when determining the user reference model 330.

Based on the referenced parameters and the user baseline profile 320, the user reference model 330 is established. According to embodiment of the present invention, the user reference model 330 can provide zone indicators that relate to deviations from the recommended guidelines by the U.S. medical associations, such as American Hospital Association (AHA), American Association of Clinical Endocrinologists (AACE), Joint National Committee on Detection, Evaluation (JNC) and World Health Organization (WHO). For example, a green zone indicates that the user is within the recommended parameters, the yellow zone indicated a deviation from the recommended parameters, which not significant, and the red zone indicates the severer deviations from the recommended parameters. The zones can be communicated to the user via remote software program 130, or clinical professional. The zones can also be updated at the same time as the user baseline profile 310 updates.

According to embodiment of the present invention, the implemented computer program 140 can also have an artificial intelligence (AI) module 350. AI module 350 allows the implemented computer program 140 to calculate the risk level for using the entire amount of use data obtained from the initial user data 310, the user baseline profile 320 and the user reference model 330. The AI module 350 can be used to determine predictive calculations of possible deterioration in user's parameters reflected in the user reference model 330 and susceptibility to new negative conditions.

A medical supervision interface module (not shown) can be used for continuous AI training. The medical supervision interface module preferably is trained by a specially dedicated team that are qualification in the field of analysis in the health care fields in order to be able to properly evaluate the predictions of the AI module 350.

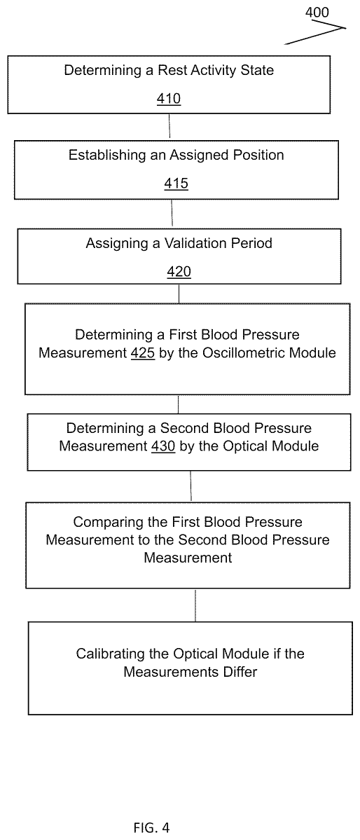

As illustrated in FIG. 4, according to embodiment of the present invention, system 100 allows for calibration 400 of blood pressure monitoring. That is, the system 100 is configured to align the blood pressure measurements of the optical module 115 with the blood pressure measurements of the oscillometric module 120. This provides substantial advantage over the known systems that usually require calibration being performed using outside monitoring devices. Embodiments of the present invention, on one hand allow for continuous real-time monitoring through the optical module 115, and on the other hand allowing calibration of the measurements from the optical module 115 by more accurate and precise second measurement that is determined by the oscillometric module 120.

As shown in FIG. 4, the system 100 first determines a rest state of the user 410. If the system 100 determines via the wearable device 110 that the user is in the rest position, then the system 100 detects whether the wearable device is located on the left hand or the right hand of the user via advance positioning sensor 290. The system 100 also establishes whether the user's arm position with the wearable device 110 is at the proper angle (i.e., an assigned position 415) as determined by the advance positioning sensor 290. The user can then establish a validation period 420 via a user interface (not shown) of the remote software application. The validation period can be between 3 to 30 days, and preferable 3 days. Upon expiration of the validation period 420, the system 100 must be recalibrated again. Once the validation period 420 is established the system 100 determines a first blood pressure measurement 425 by the oscillometric module 120. Then, the system 100 determines a second blood pressure measurement 430 by the optical module 115. Once both, the first blood pressure measurement 425 and the second blood pressure measurement 430 are established, then the first blood pressure measurement 425 is compared to the second blood pressure 430 measurement. If the second blood pressure measurement 430 is different from the first blood pressure measurement 425, the optical module 115 is calibrated based on the first blood pressure measurement.

The remote software application 130 is configured to provide a real-time feedback 180. The feedback 180 can provide information about the vital signs measurements, the results of the comparison between the actual vital signs measurements and the user reference model based on the interpretation by the computer program 14, as well as track prescriptions, medication taken and scheduling of measurements. In addition to functionalities described in this disclosure, the software application 130 can also include a SOS function, which alerts caregivers and medical personnel when there is an emergency.

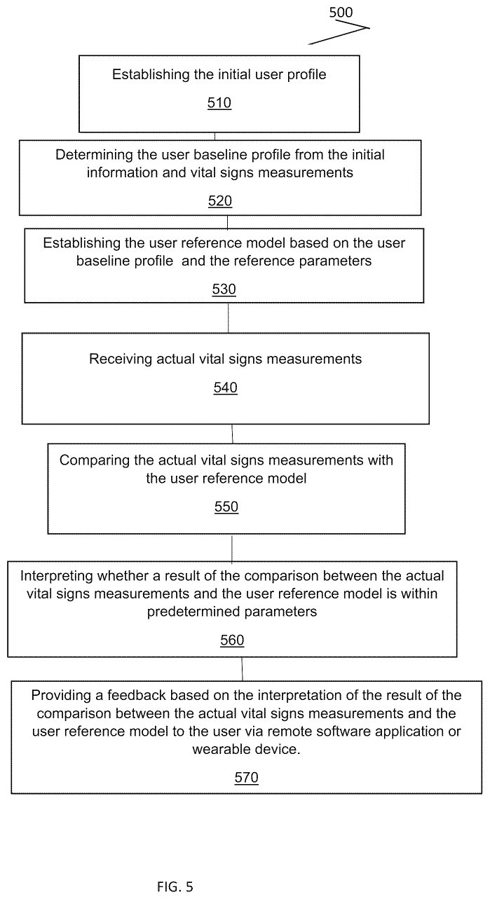

FIG. 5 is a flow diagram illustrating method 500 for measuring and monitoring vital signs of the user using system 100 in according with embodiments of the present invention. The method 500 includes accessing the wearable device 110 and accessing the computer program 140, for example through the remote software application 130. In step 510, the user either accesses or establishes the user initial profile 310 based on an initial information. In step 520, the user baseline profile 320 is determined from the initial information and vital signs measurements received from the wearable device 110 for a time period. The time period is preferably 7 days. In the step 530, the user reference model 330 based on the user baseline profile 320 and the reference parameters is established. The user reference model 330 can include zone indicators. In step 540, actual vital signs measurements are received from the wearable device 110. In step 550, the actual vital signs measurements are compared with the user reference model 330. Then, in step 560, the computer program 140 interprets whether a result of the comparison between the actual vital signs measurements and the user reference model is within predetermined parameters. Finally, in step 570, the feedback 180 based on the interpretation of the result of the comparison between the actual vital signs measurements and the user reference model is provided to the user via remote software application 130 or wearable device 110.

The foregoing detailed description of the embodiments is used to further clearly describe the features and spirit of the present invention. The foregoing description for each embodiment is not intended to limit the scope of the present invention. All kinds of modifications made to the foregoing embodiments and equivalent arrangements should fall within the protected scope of the present invention. Hence, the scope of the present invention should be explained most widely according to the claims described thereafter in connection with the detailed description, and should cover all the possibly equivalent variations and equivalent arrangements.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a memory stick, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or steps plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A calibrated system for measuring and monitoring vital signs of a user, the system comprising:
a wearable device, the wearable device comprises an optical module, an oscillometric module and an electrocardiogram (ECG) module;
at least one processor; and
a non-transitory machine-readable medium comprising instructions stored therein, which when executed by the at least one processor, cause the processor to perform operations comprising:
establishing a user initial profile based on an initial information;
determining a user baseline profile from the initial information, vital signs measurements received from the wearable device for a time period;
determining a user reference model based on the user baseline profile, the user reference model comprises zone indicators;
receiving actual vital signs measurements in real time from the wearable device;
comparing the actual vital signs measurements with the user reference model;
interpreting whether a result of the comparison between the actual vital signs measurements and the user reference model is within predetermined parameters; and
providing a feedback based on the interpretation of the result of the comparison between the actual vital signs measurements and the user reference model.

2. The system according to claim 1, wherein the wearable device further comprises an accelerometer and/or a gyroscope that are configured to determine the positioning of the wearable device during receiving the vital signs measurements by the processor and a user activity state.

3. The system according to claim 1, wherein the oscillometric module comprises:
a cuff configured to inflate using a pump and deflate using an electric valve;
a pump driver; and
a pressure sensor to determine user's systolic and diastolic blood pressure.

4. The system according to claim 1, wherein the ECG module comprises:
a first electrode;
a second electrode; and
a plurality of electric sensors configured to collect ECG datapoints that are converted into an ECG waveform by the processor,
wherein the ECG module is configured to automatically switch an ECG algorithm depending on a hand selection of the wearable device.

5. The system according to claim 4, wherein the first electrode is located on a bottom of the wearable device, the bottom being closest to the user's wrist, and the second electrode is located on a front of the wearable device.

6. The system according to claim 1, wherein the optical module comprises:
an optical sensor;
a light-emitting diode (LED); and
a photodetector.

7. The system according to claim 1, wherein the operations further comprise:
determining a rest activity state of the user;
establishing an assigned position of the wearable device;
assigning a validation period;
determining a first blood pressure measurement by the oscillometric module;
determining a second blood pressure measurement by the optical module;
comparing the first blood pressure measurement to the second blood pressure measurement; and
if the second blood pressure measurement is measurably different from the first blood pressure measurement, calibrating the optical module based on the first blood pressure measurement.

8. The system according to claim 1, wherein the vital signs measurements comprise skin temperature, blood pressure, heart rate, respiration rate, ECG pulse rate, and functional oxygen saturation.

9. The system according to claim 1, wherein the user initial profile comprises name, gender, age and a non-medical information of the user.

10. The system according to claim 1, wherein the user reference model comprises reference parameters, the reference parameters comprise guidelines, deviations inherent in a disease, anamnesis, medication treatment of the user, life style of the user, and the vital signs measurements for a predetermined period of time.

11. The system according to claim 1, wherein the zone indicators comprise normal, insignificant deviation, and severer deviation.

12. The system according to claim 1, wherein the feedback is displayed as a text, a voice, and/or an animation on a user device.

13. A computer-implemented method for measuring and monitoring vital signs of a user, the method comprising:
establishing a user initial profile based on an initial information;
determining a user baseline profile from the initial information, vital signs measurements received from a wearable device for a time period;
determining a user reference model based on the user baseline profile, the user reference model comprises zone indicators;
receiving actual vital signs measurements in real time from the wearable device;
comparing the actual vital signs measurements with the user reference model;
interpreting whether a result of the comparison between the actual vital signs measurements and the user reference model is within predetermined parameters; and
providing a feedback based on the interpretation of the result of the comparison between the actual vital signs measurements and the user reference model.

14. The method according to claim 13, wherein the wearable device comprises an optical module, an oscillometric module and an electrocardiogram (ECG) module;
a processor; and
a non-transitory machine-readable medium comprising instructions stored therein.

15. The method according to claim 14, wherein the wearable device further comprises an accelerometer and/or a gyroscope that are configured to determine the positioning of the wearable device during receiving the vital signs measurements by the processor and a user activity state.

16. The method according to claim 15 further comprising:
determining a rest activity state of the user;
establishing an assigned position of the wearable device;
assigning a validation period;
determining a first blood pressure measurement by the oscillometric module;
determining a second blood pressure measurement by the optical module;
comparing the first blood pressure measurement to the second blood pressure measurement; and
if the second blood pressure measurement is measurably different from the first blood pressure measurement, calibrating the optical module based on the first blood pressure measurement.

17. The method according to claim 13 wherein the vital signs measurements comprise skin temperature, blood pressure, heart rate, ECG, respiration rate, pulse rate, and functional oxygen saturation.

18. The method according to claim 13, wherein the user initial profile comprises name, gender, age other non-medical information of the user.

19. The method according to claim 13, wherein the user baseline profile comprises reference parameters, deviations inherent in a disease, anamnesis, medication treatment of the user, life style of the user, the vital signs measurements for a predetermined period of time.

20. The method according to claim 13, wherein the zone indicators comprise normal, insignificant deviation, and severer deviation.

21. A computer-implemented method for calibrating a blood pressure measuring device, wherein the blood pressure measuring device comprise an optical module, an oscillometric module, an accelerometer and/or a gyroscope, the method comprising:
determining a rest activity state of the user;
establishing an assigned position of the wearable device;
assigning a validation period;
determining a first blood pressure measurement by the oscillometric module;
determining a second blood pressure measurement by the optical module;
comparing the first blood pressure measurement to the second blood pressure measurement; and
if the second blood pressure measurement is measurably different from the first blood pressure measurement, calibrating the optical module based on the first blood pressure measurement.
